SEQUENCE LISTING (1 sequence)

SEQ ID NO: 1 - protein, 791 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

(19) United States
(12) Patent Application Publication
(10) Pub. No.: US 2024/0299506 A1
(43) Pub. Date: Sep. 12, 2024

(54) WOUND HEALING

(71) Applicant: GRIFOLS WORLDWIDE OPERATIONS LIMITED, DUBLIN (IE)

(72) Inventor: Valery NOVOKHATNY, Research Triangle Park, NC (US)

(21) Appl. No.: 18/252,995

(22) PCT Filed: Nov. 23, 2021
(86) PCT No.: PCT/EP2021/082691

§ 371 Date: May 15, 2023

Related U.S. Application Data
(60) Provisional application No. 63/118,473, filed on Nov. 25, 2020.

(51) Int. Cl. A61K 38/48; A61P 17/02

(52) U.S. Cl. CPC A61K 38/484; A61P 17/02

(57) ABSTRACT

Methods and compositions for the treatment of wounds and ulcers in patients, in particular those patients suffering from chronic, non-healing wounds and ulcers. Fibrinogenases, such as plasmin show utility in decreasing plasma and or blood viscosity resulting in improved wound and ulcer healing in patients. The fibrinogenase may be one selected from the group consisting of an α-fibrinogenase, a β-fibrinogenase, a γ-fibrinogenase, a metallo-α-fibrinogenase, allium-α-fibrinogenase, a plasmin, and combination thereof.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is DURC5_028 APC_SL. TXT, the date of creation of the ASCII text file is May 12, 2023, and the size of the ASCII text file is 7.0 KB.

### FIELD OF THE INVENTION

The present invention relates to methods and compositions for the treatment of wounds and ulcers in patients, in particular those patients suffering from chronic, non-healing wounds and ulcers. The invention details a heretofore unreported utility of a fibrinogenase, such as the serine protease plasmin in the treatment of said wounds and ulcers.

### BACKGROUND

In normal skin, the epidermis and dermis exist in steady-state equilibrium, forming a protective barrier against the external environment. Once the protective barrier is broken, the process of wound healing is immediately set in motion comprising a set of complex biochemical events taking place in a closely orchestrated cascade to repair the damage. The classic model of wound healing is divided into four sequential, yet overlapping phases: (1) haemostasis, (2) inflammatory, (3) proliferative, and (4) remodelling.

In stage 1 homeostasis, platelets (thrombocytes) are recruited to the injury site to form a fibrin clot to prevent active bleeding. In the inflammatory phase, bacteria and debris are phagocytosed and removed, and factors are released that cause the migration and division of cells involved in the proliferative phase. The proliferative phase is characterized by angiogenesis, collagen deposition, granulation tissue formation, epithelialization, and wound contraction. In fibroplasia and granulation tissue formation fibroblasts grow and form a new, provisional extracellular matrix (ECM) by providing collagen and fibronectin. Concurrently, re-epithelialization of the epidermis occurs in which epithelial cells proliferate and ‘crawl’ atop the wound bed providing cover for the new tissue. As the healing process continues, the wound is made smaller by the action of myofibroblasts. In the final remodelling phase, type III collagen, which is prevalent during proliferation, is replaced by type I collagen. Originally disorganized collagen fibres are rearranged, cross-linked, and aligned along tension lines.

The wound healing process is sensitive and susceptible to interruption leading to the formation of non-healing chronic wounds. For healthy adults, the four wound healing stages progress naturally and a wound can be healed within 2-3 weeks. For those less fortunate, the body's natural healing process can be interrupted or diminished resulting in wounds healing much slower. These wounds are called chronic wounds (wounds that do not heal in greater than four weeks despite normal treatment) and are most common in people with diabetes, high blood pressure, obesity and other vascular diseases. If not cared for or treated, chronic wounds can lead to pain, infection, disability, and possibly amputation of the affected limb. Non-healing wounds represent a significant burden to both the patient and the medical system. It is estimated that chronic non-healing wounds affect nearly 15% of Medicare beneficiaries in the U.S. at an annual cost to Medicare of $28.1 to $31.7 billion. See Nussbaun S R et al., An Economic Evaluation of the Impact, Cost, and Medicare Policy Implications of Chronic Nonhealing Wounds, Value in Health, 2018, V. 21, Issue 1, 27-32.

Strategies employed by medical professionals to accelerate wound healing include compression techniques, revascularization surgeries to treat arterial ulcers; venous surgical interventions for venous ulcers, negative-pressure wound therapy, and hyperbaric oxygenation. In terms of pharmacological intervention very few biologically active substances have reached clinical practice and pharmacological intervention is not widely used for the direct treatment of chronic wounds.

The plasminogen activator system is a proteolytic system in which the proteolytically inert zymogen plasminogen is converted to the active protease plasmin by the action of tissue-type plasminogen activator (tPA) or urokinase-type plasminogen activator (uPA). The plasminogen activator system not only plays a role in fibrinolysis but also in many tissue-remodelling processes, including wound healing by providing space for cells to proliferate into. See Romer J, Bugge T H, Pyke C, et al. Impaired wound healing in mice with a disrupted plasminogen gene. Nat Med. 1996:2(3):287-292.

Plasminogen is adsorbed to specific sites on fibrin whereupon it is acted on by tPA leading to the rapid, localised formation of plasmin appropriate for the cleavage of fibrin and other proteins of the extracellular matrix. Plasminogen has also been shown to play a critical role in the activation of intracellular signalling events and in the generation of inflammatory responses in the wound healing process, see Ny et al., Blood. 2012; 119(24):5879-5887. Ny and co-workers describe plasminogen as a key proinflammatory regulator and signalling molecule that accelerates the healing of acute and diabetic wounds. Early in the healing process, circulating plasminogen is bound to inflammatory cells and is transported to the wound area, thus increasing the level of plasminogen locally, which in turn further leads to the induction of cytokines, intracellular signalling events, and the potentiation of an early inflammatory response. Administration of additional plasminogen is reported to accelerate the healing of acute burn wounds in wild-type mice, and also improves the healing of wounds in a diabetic mouse model.

Based on its plasma stability/half-life and its role in intracellular signalling/inflammation pharmacological intervention with exogenous plasminogen, administered locally in proximity to the wound, has been the focus research groups in the area of wound healing.

### DESCRIPTION OF THE INVENTION

The words “comprises/comprising” and the words “having/including” when used herein with reference to the present invention are used to specify the presence of stated features, integers, steps or components but do not preclude the presence or addition of one or more other features, integers, steps, components or groups thereof.

It should be appreciated by those skilled in the art that the specific embodiments disclosed herein should not be read in isolation, and that the present specification intends for the disclosed embodiments to be read in combination with one another as opposed to individually. As such, each embodiment may serve as a basis for modifying or limiting other embodiments disclosed herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “10 to 100” should be interpreted to include not only the explicitly recited values of 10 to 100, but also include individual value and sub-ranges within the indicated range. Thus, included in this numerical range are individual values such as 10, 11, 12, 13 . . . 97, 98, 99, 100 and sub-ranges such as from 10 to 40, from 25 to 40 and 50 to 60, etc. This same principle applies to ranges reciting only one numerical value, such as “at least 10”. Furthermore, such an interpretation should apply regardless of the breadth of the range or the characteristics being described.

### Treatment of the Invention

In a first aspect, the present invention provides for a method of reducing blood viscosity in a patient in need thereof, the method comprising parenteral administration of a therapeutically effective amount of a fibrinogenase to the patient. The skilled person will appreciate that blood viscosity can be measured as systolic blood viscosity or diastolic blood viscosity, and that both modalities are applicable to the present invention. In one embodiment, the present invention provides for a method of reducing diastolic blood viscosity in a patient in need thereof.

As used herein, the term “parenteral administration” refers to a route of administration that results in the drug being absorbed outside the gastrointestinal tract. Non-limiting examples of parenteral administration include intravenous, intramuscular, intraperitoneal, and subcutaneous. In one embodiment, the fibrinogenase is administered intravenously.

In one embodiment, the patient in need of a reduction in blood viscosity (for example, diastolic blood viscosity) suffers from a wound or ulcer. The wound or ulcer may be a chronic wound or ulcer.

As used herein, the term “ulcer” refers to a tissue lesion in which the primary tissue breakdown is internal within the patient, e.g., the lesion is caused by an underlying disease or other internal reason. Non-limiting examples of ulcers include:
venous leg ulcers caused by cardiovascular disease or venous insufficiency, neuropathic/diabetic (foot) ulcers caused by diabetes mellitus, decubitus/pressure ulcers caused by immobility or vascular stasis/swelling in a particular area, and
arterial or ischemic ulcers caused by poor perfusion (delivery of nutrient-rich blood) to the extremities.

As used herein, the term “wound” refers to a tissue lesion in which the primary tissue breakdown in external. Non-limiting examples of wounds include trauma wounds caused by an accident/external force, surgically induced wounds caused by incision, and burns caused by external heat.

Wounds and ulcers are defined as being acute or chronic depending upon how healing progresses. By “acute” it is meant that the wound or ulcer progress through the normal stages of wound healing and exhibits definite signs of healing within four weeks. By “chronic” it is meant that the wound or ulcer does not progress normally through the stages of healing and does not show evidence of healing within four weeks.

In a second aspect, the present invention provides for a method for the treatment of a wound or ulcer in a patient in need thereof, the method comprising parenteral administration of a therapeutically effective amount of a fibrinogenase to the patient.

In one embodiment, the fibrinogenase is administered over a dosing period, wherein at one or more time-points during the dosing period the patient's plasma viscosity decreases by at least about 1.0% compared to the patient's pre-treatment plasma viscosity level. For example, the patient's plasma viscosity may decrease by about at least 1.5% compared to the patient's pre-treatment plasma viscosity level. In one embodiment, the patient's plasma viscosity may decrease by about at least 2.0% compared to the patient's pre-treatment plasma viscosity level. In another embodiment, the patient's plasma viscosity may decrease by about at least 2.5% compared to the patient's pre-treatment plasma viscosity level. In a further embodiment, the patient's plasma viscosity may decrease by about at least 3.0% compared to the patient's pre-treatment plasma viscosity level. In yet a further embodiment, the patient's plasma viscosity may decrease by about at least 3.5% compared to the patient's pre-treatment plasma viscosity level. For example, the patient's plasma viscosity may decrease by about at least 4.0% compared to the patient's pre-treatment plasma viscosity level. In one embodiment, the patient's plasma viscosity may decrease by about at least 5.0% compared to the patient's pre-treatment plasma viscosity level.

In one embodiment, the fibrinogenase is administered over a dosing period, wherein at one or more time-points during the dosing period the patient's plasma viscosity decreases in the range of about 1.0% to about 20.0% compared to the patient's pre-treatment plasma viscosity level. In one embodiment, the patient's plasma viscosity may decrease in the range of about 1.0% to about 15.0% compared to the patient's pre-treatment plasma viscosity level. In a further embodiment, the patient's plasma viscosity may decrease in the range of about 1.0% to about 10.0% compared to the patient's pre-treatment plasma viscosity level. In yet a further embodiment, the patient's plasma viscosity may decrease in the range of about 1.0% to about 5.0% compared to the patient's pre-treatment plasma viscosity level. For example, the patient's plasma viscosity may decrease in the range of about 2.5% to about 20.0% compared to the patient's pre-treatment plasma viscosity level. In one embodiment, the patient's plasma viscosity may decrease in the range of about 2.5% to about 15.0% compared to the patient's pre-treatment plasma viscosity level. In a further embodiment, the patient's plasma viscosity may decrease in the range of about 2.5% to about 10% compared to the patient's pre-treatment plasma viscosity level. In yet a further embodiment, the patient's plasma viscosity may decrease in the range of about 2.5% to about 7.5% compared to the patient's pre-treatment plasma viscosity level. For example, the patient's plasma viscosity may decrease in the range of about 5.0% to about 20.0% compared to the patient's pre-treatment plasma viscosity level. In one embodiment, the patient's plasma viscosity may decrease in the range of about 5.0% to about 15.0% compared to the patient's pre-treatment plasma viscosity level. In a further embodiment, the patient's plasma viscosity may decrease in the range of about 5.0% to about 10% compared to the patient's pre-treatment plasma viscosity level.

The principal determinants of whole blood viscosity are: (1) hematocrit; (2) red blood cell deformability (i.e., the structural response of red cells to applied forces); and (3) the viscosity of plasma. This viscosity of plasma is directly proportional to the concentrations of certain plasma proteins such as immunoglobulins, lipoproteins, and fibrinogen. Without wishing to be bound by theory, the present inventors postulate that by administering a fibrinogenase to the patient plasma viscosity can be decreased, translating into an attendant decrease in whole blood viscosity. In turn, blood circulation to the wounds or ulcers is improved, thereby increasing the rate at which the wound or ulcer heals.

As will be appreciated by a person of skill in the art there are a number of ways of measuring plasma viscosity. For example, plasma viscosity may be measured using a capillary viscometer, a falling body viscometer, or a rotational viscometer.

Non-limiting examples of a capillary viscometer include the Ostwald U-tube Viscometer, and the Harkness Viscometer. Non-limiting examples of a falling body viscometer include the Stoney Brook Falling Needle Viscometer, and Electromagnetic Viscometers such as the VISCOlab series developed and marketed by Cambridge Viscosity. Suitable, non-limiting examples of a rotational viscometer include a Cone and Plate Viscometer, and a Brookfield Viscometer.

It will be appreciated by those skilled in the art that the methodology/equipment utilised to measure plasma viscosity is not determinative of the scope of the present invention. Mammalian plasma has a density of very close to 1.0. As such, viscometers measuring either the kinematic viscosity or dynamic viscosity are utilisable to measure the reduction in plasma viscosity in accordance with the method of the present invention. Any viscosity measuring methodology can be applied to the measurement of plasma viscosity, provided the same methodology is utilised to measure the patient's pre- and post-treatment viscosity levels.

In one particular embodiment, the dynamic viscosity of the patient's plasma following treatment with a fibrinogenase is measured using an Electromagnetic Viscometer at 22° C. and 1 atm pressure. For example, the dynamic viscosity of the patient's plasma following treatment with the fibrinogenase may be tested using a VISCOlab 4000 (Cambridge Viscosity) laboratory viscometer at 22° C. and 1 atm pressure.

In a third aspect, the present invention provides for a method for the treatment of a wound or ulcer in a patient in need thereof, the method comprising parenteral administration of a therapeutically effective amount of a fibrinogenase to the patient over a dosing period, wherein at one or more time-points during the dosing period fibrinogen levels in the patient's plasma decrease by at least about 5% compared to the patient's pre-treatment fibrinogen level.

In one embodiment, the fibrinogen levels in the patient's plasma may decrease by at least about 10% compared to the patient's pre-treatment fibrinogen level. In one embodiment, the fibrinogen levels in the patient's plasma may decrease by at least about 20% compared to the patient's pre-treatment fibrinogen level. In one embodiment, the fibrinogen levels in the patient's plasma may decrease by at least about 25% compared to the patient's pre-treatment fibrinogen level. In one embodiment, the fibrinogen levels in the patient's plasma may decrease by at least about 30% compared to the patient's pre-treatment fibrinogen level. In one embodiment, the fibrinogen levels in the patient's plasma may decrease by at least about 35% compared to the patient's pre-treatment fibrinogen level. In one embodiment, the fibrinogen levels in the patient's plasma may decrease by at least about 40% compared to the patient's pre-treatment fibrinogen level. In one embodiment, the fibrinogen levels in the patient's plasma may decrease by at least about 45% compared to the patient's pre-treatment fibrinogen level. In one embodiment, the fibrinogen levels in the patient's plasma may decrease by at least about 50% compared to the patient's pre-treatment fibrinogen level.

In one embodiment, the fibrinogenase is administered over a dosing period, wherein at one or more time-points during the dosing period the fibrinogen levels in the patient's plasma decrease in the range of about 5% to about 70% compared to the patient's pre-treatment fibrinogen level. In one embodiment, the fibrinogen levels in the patient's plasma may decrease in the range of about 15% to about 70% compared to the patient's pre-treatment fibrinogen level. In one embodiment, the fibrinogen levels in the patient's plasma may decrease in the range of about 25% to about 70% compared to the patient's pre-treatment fibrinogen level. In one embodiment, the fibrinogen levels in the patient's plasma may decrease in the range of about 35% to about 70% compared to the patient's pre-treatment fibrinogen level. In one embodiment, the fibrinogen levels in the patient's plasma may decrease in the range of about 45% to about 70% compared to the patient's pre-treatment fibrinogen level.

In one embodiment, the fibrinogen levels in the patient's plasma may decrease in the range of about 10% to about 65% compared to the patient's pre-treatment fibrinogen level. In one embodiment, the fibrinogen levels in the patient's plasma may decrease in the range of about 10% to about 60% compared to the patient's pre-treatment fibrinogen level. In one embodiment, the fibrinogen levels in the patient's plasma may decrease in the range of about 10% to about 55% compared to the patient's pre-treatment fibrinogen level. In one embodiment, the fibrinogen levels in the patient's plasma may decrease in the range of about 10% to about 50% compared to the patient's pre-treatment fibrinogen level.

As will be appreciated by a person of skill in the art, there are multiple methods utilised to clinically measure fibrinogen levels in plasma. Non limiting examples include:
Total clottable fibrinogen (Blombäck and Blombäck methodology),
Clotting time assay/Clauss assay,
Radial immunodiffusion according to Mancini et al.,
Total amount of clottable fibrinogen by means of turbidimetric assay according to Ellis and Stransky,
Immunological assays,
ChromotimeSystem, and
Prothrombin time (PT)-derived fibrinogen assay on ACL coagulometer.

All these assays are common general knowledge and familiar to the person of skill in the art, and multiple reviews abound in the literature about the various techniques. Two such reviews are Palareti G. et al. Fibrinogen assays: a collaborative study of six different methods. C.I.S.M.E.L. Clin Chem. 1991 May: 37(5):714-9, and Mackie et al., Guidelines on fibrinogen assays. British Journal of Haematology, 2003,121,396-404 the contents of which are incorporated herein by reference.

It will be appreciated by those skilled in the art that the assay utilised to measure plasma fibrinogen levels is not determinative of the scope of the present invention. The present invention is concerned with the relative decrease in plasma fibrinogen levels before and after treatment. Any fibrinogen assay methodology can be applied to the measurement of plasma fibrinogen levels, provided the same assay is utilised to measure the patient's pre- and post-treatment fibrinogen levels.

In one embodiment, the plasma fibrinogen levels are determined by the clotting time/Clauss methodology, see Von Clauss A. Gerinnungsphysiologische Schnellmethode zur Bestimmung des Fibrunogens. Acta Haematol 1957:17:237-246, the contents of which are incorporated herein by reference.

Briefly, the Clauss assay is a fibrinogen activity test. A high concentration of thrombin (ranging from 35 to 200 U/ml, but typically about 100 U/ml) is added to dilute test plasma sample and the clotting time is measured. The test result is compared with a calibration curve prepared by clotting a series of dilutions of a reference plasma sample of known fibrinogen concentration, and a result in g/l is obtained. The time it takes for a clot to form directly correlates with the amount of active fibrinogen that is present.

In one embodiment, the treatment methods of the present invention aim to improve the healing of a venous leg ulcer. In one embodiment, the treatment methods of the present invention aim to improve the healing of a neuropathic or diabetic ulcer. In one embodiment, the treatment methods of the present invention aim to improve the healing of a decubitus or pressure ulcer. In one embodiment, the treatment methods of the present invention aim to improve the healing of an arterial or ischemic ulcer.

In some embodiments, the treatment methods of the present invention are aimed at improving the healing of chronic wounds and ulcers. For example, chronic venous leg ulcers, chronic diabetic ulcers, chronic pressure ulcers, chronic ischemic ulcers, and combinations thereof.

### Fibrinogenase

Unexpectedly, the present inventors have discovered that systemic administration of a fibrinogenase was successful in improving the treatment of wounds and/or ulcers in patients. Without wishing to be bound by theory, the present inventors postulate that by administering a fibrinogenase to a patient suffering with a (chronic) wound or ulcer the protease would degrade and reduce concentrations of fibrinogen in the patient's plasma. In turn, the patient's blood viscosity would decrease so as to improve micro-circulation to the wound or ulcer, thereby increasing the rate at which the wound or ulcer heals.

By “protease” the present specification means a molecule that breaks down large proteins into smaller proteins and/or peptides.

By “fibrinogenase” it is meant a protease that can break down the protein fibrinogen. Fibrinogenases may be classified as α, β and γ-fibrinogenases based on their specificity for cleaving fibrinogen polypeptide chains. The fibrinogenase used in the method of the present invention may be selected from the group consisting of an α-fibrinogenase, a β-fibrinogenase, a γ-fibrinogenase, and combinations thereof. Fibrinogenases finding utility in the present invention may be manufactured recombinantly or isolated from a natural source.

In one embodiment, the fibrinogenase is selected from the group consisting of a metallo-α-fibrinogenase, allium-α-fibrinogenase, and combinations thereof.

In a particular embodiment, the fibrinogenase utilised in the method of the present invention is a plasmin. Plasmins finding utility in the present invention may be manufactured recombinantly or derived from plasminogen isolated from a natural source, such as isolated from human plasma.

It should be appreciated by those skilled in the art that the specific embodiments disclosed within the previous paragraphs should not be read in isolation, and that the present specification intends for these embodiments to be disclosed in combination with other embodiments as opposed to being disclosed individually.

### Plasmin

Native circulating human plasminogen, as set forth in SEQ ID NO: 1, is a single-chain protein containing 791 amino acid residues with 24 intra-chain disulfide bridges, 5 kringle domains, a serine protease domain, and an preactivation peptide (PAP). The locations of these domains with respect to SEQ ID NO: 1 are outlined in Table 1 infra.

TABLE 1

| Residues (per SEQ ID No: 1) | Description | Length |
|---|---|---|
| 1-79 | PAP Domain | 79 |
| 84-162 | Kringle 1 | 79 |
| 165-243 | Kringle 2 | 79 |
| 275-333 | Kringle 3 | 78 |
| 358-435 | Kringle 4 | 78 |
| 462-541 | Kringle 5 | 80 |
| 562-789 | S1 Peptidase | 228 |

Plasminogen is produced as Glu-Plasminogen and Lys-Plasminogen depending on whether the N-terminal amino acid is either glutamic acid or lysine. Glu-Plasminogen is composed of the entire amino acid sequence designated by the gene sequence (excluding the precursor peptide). Lys-Plasminogen is the result of a cleavage of Glu-Plasminogen between Lys-77 and Lys-78. Glu-Plasminogen is the dominant form of plasminogen present in human plasma.

Once it has been secreted into plasma, plasminogen can be converted into plasmin by the action of tissue-type plasminogen activator (t-PA) and urokinase plasminogen activator (u-PA). t-Pa/u-PA cleave the Arg561-Val562 peptide bond in the plasminogen zymogen. The resulting plasmin molecule is a two-chain, disulfide-linked serine protease with trypsin-like specificity (cleaves after Lys and Arg).

The amino-terminal heavy chain of plasmin is composed of the five kringle domains, each containing approximately 80 amino acid residues. The kringle domains are responsible for the interactions of plasmin with other proteins, such as polymeric fibrin and the plasmin inhibitor aλ-antiplasmin.

The C-terminal light chain of plasmin is a typical serine protease, homologous to trypsin and containing the classic serine protease catalytic triad: His603, Asp646, and Ser741.

As used herein, the term “plasmin” is to be construed as meaning a therapeutically effective amount of:
a wild type (human) plasmin protein,
a functional mutant thereof,
a functional fragment thereof, or
combinations thereof.

FIG. 1 discloses a schematic of a number of plasmin variants and mutants within the scope of the present invention. Mutants having various permutations of kringle domains 1-5 tethered to the serine protease component are within the scope of the invention. Minor variations in the amino acid sequence are irrelevant, provided the motifs outlined in FIG. 1 are maintained. The information outlined in SEQ ID NO: 1, Table 1 and FIG. 1 affords the skilled person with sufficient direction and clarity as to which plasmin mutants fall within the scope of the present invention.

For example “plasmin” includes, but is not limited to:
(human) Glu-plasmin,
(human) Lys-plasmin,
midi-plasmin,
mini-plasmin,
micro-plasmin (for example, as disclosed in U.S. Pat. No. 4,774,087 incorporated herein by reference in its entirety, or in the commercialised plasmin mutant Ocriplasmin),
deltaplasmin (for example, as disclosed in U.S. Pat. No. 8,420,079 incorporated herein by reference in its entirety), and
combinations thereof.

In one embodiment, the plasmin is (human) glu-plasmin. In some embodiments, the plasmin is (human) lys-plasmin. In other embodiments, the plasmin is a mixture of glu- and lys-plasmin.

The unforeseen efficacy of plasmin in the treatment of wounds and ulcers by means of reduction in plasmin viscosity is particularly surprising given that systemic/intravenous thrombolytic therapy with plasmin had largely been viewed as ineffective, see Marder V J. Historical perspective and future direction of thrombolysis research: the re-discovery of plasmin. J Thromb Haemost. 2011; 9 Suppl 1:364-373. Free plasmin released into the plasma (unbound to fibrin) is instantly neutralised by circulating protease inhibitors such as a2-antiplasmin, thereby rendering it inactive.

Moreover, the ability of plasmin to elicit a non-localised effect on would/ulcer healing is entirely unexpected. The prevailing scientific opinion is that plasmin's role in wound healing is subordinated to that of plasminogen because plasmin's effects are strictly localised in degrading fibrin in the remodelling phase of the wound healing process. Plasminogen is the preferred candidate for pharmacological intervention owing to its ability to activate intracellular signalling events, generate an improved inflammatory response, and its longer plasma half-life.

It should be appreciated by those skilled in the art that the specific embodiments disclosed within the previous paragraphs should not be read in isolation, and that the present specification intends for these embodiments to be disclosed in combination with other embodiments as opposed to being disclosed individually. For example, each of the embodiments disclosed in said paragraphs is to be read as being explicitly combined with each of the embodiments disclosed in paragraphs from the Treatment of the Invention section to those of the section immediately before to the present one, or any permutation of 2 or more of the embodiments disclosed therein.

### Dosing

In one embodiment of the method of the present invention the fibrinogenase may be administered to the patient in at least one dose of a concentration of from about 1 mg/kg to about 100 mg/kg. For example, from about 1 mg/kg to about 50 mg/kg, such as from about 1 mg/kg to about 30 mg/kg, for example from about 1 mg/kg to about 10 mg/kg.

In some embodiments of the method of the present invention the fibrinogenase may be administered to the patient in at least one dose of a concentration of from about 5 mg/kg to about 30 mg/kg, for example from about 5 mg/kg to about 20 mg/kg, such as from about 5 mg/kg to about 10 mg/kg.

In some embodiments of the method of the present invention the fibrinogenase may be administered to the patient in at least one dose of a concentration of from about 2 mg/kg to about 20 mg/kg, for example from about 4 mg/kg to about 16 mg/kg, such as from about 6 mg/kg to about 12 mg/kg.

In yet further embodiments of the method of the present invention the fibrinogenase may be administered to the patient in at least one dose of a concentration of from about 2 mg/kg to about 10 mg/kg, for example from about 4 mg/kg to about 10 mg/kg, such as from about 6 mg/kg to about 10 mg/kg.

The method of the present invention also provides for the administration of the fibrinogenase to the patient in need thereof as part of a multiple dosing regimen.

For example, the fibrinogenase can be administered at initial dose of about 5 mg/kg to about 30 mg/kg on day 1 of an administration period, followed by about 5 mg/kg to about 30 mg/kg per dose during a multiple dosing period. For example, at initial dose of about 5 mg/kg to about 20 mg/kg on day 1 of an administration period, followed by about 5 mg/kg to about 20 mg/kg per dose during a multiple dosing period. For example, at initial dose of about 5 mg/kg to about 15 mg/kg on day 1 of an administration period, followed by about 5 mg/kg to about 15 mg/kg per dose during a multiple dosing period. For example, at initial dose of about 6 mg/kg to about 12 mg/kg on day 1 of an administration period, followed by about 6 mg/kg to about 12 mg/kg per dose during a multiple dosing period.

The multiple dosing period may comprise from about 3 to about 30 administrations up to a total cumulative dose. The multiple dosing period may be from about 1 to about 10 weeks. The multiple portion doses may be administered at intervals of from about 1 day to about 30 days.

The multiple dosing period may comprise from about 3 to about 15 administrations up to a total cumulative dose. The multiple dosing period may be from about 1 to about 5 weeks. The multiple portion doses may be administered at intervals of from about 1 day to about 10 days.

It should be appreciated by those skilled in the art that the specific embodiments disclosed within the previous paragraphs should not be read in isolation, and that the present specification intends for these embodiments to be disclosed in combination with other embodiments as opposed to being disclosed individually. For example, each of the embodiments disclosed in said paragraphs is to be read as being explicitly combined with each of the embodiments disclosed in paragraphs from the Treatment of the Invention section to those of the section immediately before to the present one, or any permutation of 2 or more of the embodiments disclosed therein.

By way of example, all the dosing embodiments disclosed in paragraphs belonging to the present section apply mutatis mutandis where the fibrinogenase is a plasmin. Similarly, all the dosing embodiments disclosed in said paragraphs are to be read as being directly applicable to a plasmin selected from the group consisting of Glu-plasmin, Lys-plasmin, midi-plasmin, mini-plasmin, micro-plasmin, deltaplasmin, and combinations thereof. By way of illustration, all the dosing embodiments disclosed in said paragraphs are to be read as being directly applicable to a plasmin selected from the group consisting of Glu-plasmin, Lys-plasmin, and combinations thereof.

### Pharmaceutical Compositions of the Invention

The treatment methods of the present invention provide for the fibrinogenase to be administered to the patient as a component of a pharmaceutical composition, comprising at least one pharmaceutically acceptable carrier. The at least one pharmaceutically acceptable carrier may be chosen from adjuvants and vehicles. The at least one pharmaceutically acceptable carrier includes any and all solvents, diluents, other liquid vehicles, dispersion aids, suspension aids, surface active agents, isotonic agents, thickening agents, emulsifying agents, preservatives, as suited to the particular dosage form desired.

Suitable carriers are described in Remington: The Science and Practice of Pharmacy, 21 st edition. 2005, ed. D. B. Troy, Lippincott Williams & Wilkins, Philadelphia, and Encyclopedia of Pharmaceutical Technology, eds. J. Swarbrick and J. C. Boylan, 1988-1999 Marchcel Dekker, New York, the contents of which are incorporated herein by reference. Preferred examples of such carriers or diluents include, but are not limited to, water, saline, Ringer's solutions, glycols, dextrose solution, buffered solutions (such as phosphates, glycine, sorbic acid, and potassium sorbate) and 5% human serum albumin. Liposomes and non-aqueous vehicles such as glyceride mixtures of saturated vegetable fatty acids, and fixed oils (such as peanut oil, cottonseed oil, safflower oil, sesame oil, olive oil, corn oil and soybean oil) may also be used depending on the route of administration.

The pharmaceutical compositions utilised in the method of the present invention may be formulated for parenteral administration. The pharmaceutical composition may be enclosed in ampoules, disposable syringes, sealed bags, or multiple dose vials made of glass or plastic.

In a one embodiment, the pharmaceutical compositions of the invention are formulated for administration as an intravenous injection. The formulations can be administered continuously by infusion or by bolus injection.

The pharmaceutical compositions of the present invention may be presented as a unit dosage unit form, i.e. as physically discrete units intended as unitary dosages for the subject to be treated.

Sterile injectable solutions of the pharmaceutical composition of the present invention can be prepared by incorporating the active molecule in the required amount in an appropriate solvent with one or a combination of ingredients followed by filtered sterilization.

In one embodiment, the fibrinogenase (for example a plasmin) may be formulated as a lyophilised powder for reconstitution. In the case of sterile powders for reconstitution as sterile injectable solutions, methods of preparation include vacuum drying and freeze-drying that provide a powder of the active ingredient plus any additional desired ingredient from a previously sterile-filtered solution thereof.

Except insofar as any conventional media or agent is incompatible with the active molecules, and/or route of administration of the present invention use thereof in the compositions is contemplated to be within the scope of the present invention.

Where the fibrinogenase utilised in the method of the present invention is a plasmin, the pharmaceutical composition utilised in the method of the present invention may have an acidic pH. For example, the pharmaceutical composition may have a pH of between about 2.5 and about 4. Within the context of this specification, pH measurements are deemed to be taken in water at 25° C.

In one embodiment, pharmaceutical compositions containing a plasmin may additionally contain a buffer to maintain the acidic pH. In one embodiment, the buffer may be selected from the group consisting of a carboxylic acid, at least one amino acid, a derivative of the at least one amino acid, a dipeptide, an oligopeptide which includes the at least one amino acid, and combinations thereof. For example, the buffer may be selected from formic acid, acetic acid, citric acid, hydrochloric acid, lactic acid, malic acid, tartaric acid, benzoic acid, serine, threonine, methionine, glutamine, alanine, glycine, isoleucine, valine, alanine, aspartic acid, derivatives thereof, and combinations thereof.

Pharmaceutical compositions of the present invention containing a plasmin may further comprise at least one stabilizing agent. The stabilizing agent may be a pharmaceutically acceptable amino acid or a carbohydrate including, but not limited to, monosaccharides, disaccharides, polysaccharides, and polyhydric alcohols. For example, pharmaceutically acceptable carbohydrate stabilizers contemplated to be within the scope of the present invention include sugars such as, but not limited to, sucrose, glucose, fructose, lactose, trehalose, maltose and mannose, and sugar alcohols including, but not limited to, sorbitol and mannitol. Contemplated within the scope of the present invention are polysaccharides such as, but not limited to, dextrins, dextrans, glycogen, starches and celluloses, or any combination thereof pharmaceutically acceptable to a human or animal patient.

In one embodiment, the stabilizing agent may be selected from the group consisting of glycerol, niacinamide, glucosamine, thiamine, citrulline and inorganic salts such as, but not limited to, sodium chloride, potassium chloride, magnesium chloride, calcium chloride, or any combination thereof.

In one embodiment, the fibrinogenase (for example a plasmin) may constitute at least 20% by weight of the total protein content of the pharmaceutical composition of the present invention. For example, the plasma protein protease may constitute greater than or equal to about 30%, 40%, 50%, 60%, 70%, 75%, 80%, 85%, 90%, 93%, 95%, 96%, 97%, 98%, or 99% by weight of the total protein content of the pharmaceutical composition of the present invention.

It should be appreciated by those skilled in the art that the specific embodiments disclosed within the previous paragraphs should not be read in isolation, and that the present specification intends for these embodiments to be disclosed in combination with other embodiments as opposed to being disclosed individually. For example, each of the embodiments disclosed in said paragraphs is to be read as being explicitly combined with each of the embodiments disclosed in paragraphs from the Treatment of the Invention section to those of the section immediately before to the present one, or any permutation of 2 or more of the embodiments disclosed therein.

By way of example, each of the embodiments disclosed in the previous paragraphs applies mutatis mutandis where the fibrinogenase is a plasmin. Similarly, all the embodiments disclosed in said paragraphs are to be read as being directly applicable to a plasmin selected from the group consisting of Glu-plasmin, Lys-plasmin, midi-plasmin, mini-plasmin, micro-plasmin, deltaplasmin, and combinations thereof. By way of illustration, all the embodiments disclosed in said paragraphs are to be read as being directly applicable to a plasmin selected from the group consisting of Glu-plasmin, Lys-plasmin, and combinations thereof.

### BRIEF DESCRIPTION OF THE DRAWINGS

Additional features and advantages of the present invention will be made clearer in the appended drawings, in which:

FIG. 1 is a schematic of a number of plasmin variants and mutants within the scope of the present invention;

FIG. 2 plots the effects of plasmin and variants thereof on fibrinogen concentrations in-vitro;

FIG. 3 plots the effect of full-length plasmin on plasma viscosity at varying concentrations on pooled human plasma samples;

FIG. 4 illustrates a wound healing study protocol in accordance with the present invention;

FIG. 5 plots the effects of plasmin and control molecules on fibrinogen concentrations in rats over the study period;

FIG. 6 plots the effects of plasmin and control molecules on wound size reduction in rats over the study period;

FIGS. 7A to 7C demonstrate the effect of plasmin and controls on wound histology in rats; and

FIG. 8 illustrates the effect plasmin and control molecules on wound histology in rats.

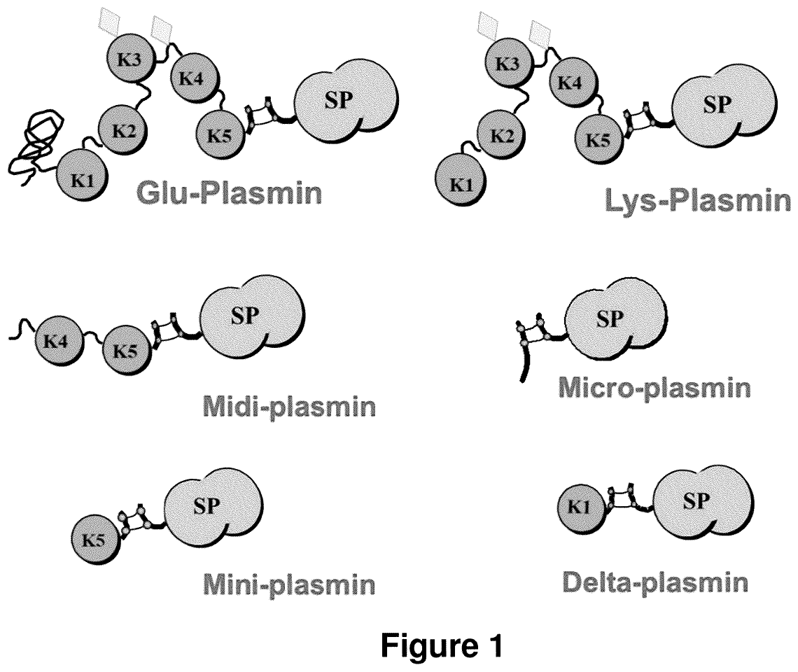

### DETAILED EXAMPLES OF THE INVENTION

It should be readily apparent to one of ordinary skill in the art that the examples disclosed herein below represent generalised examples only, and that other arrangements and methods capable of reproducing the invention are possible and are embraced by the present invention.

### Example 1—the Effect of Plasmin and Variants Thereof on Fibrinogen Concentrations In-Vitro

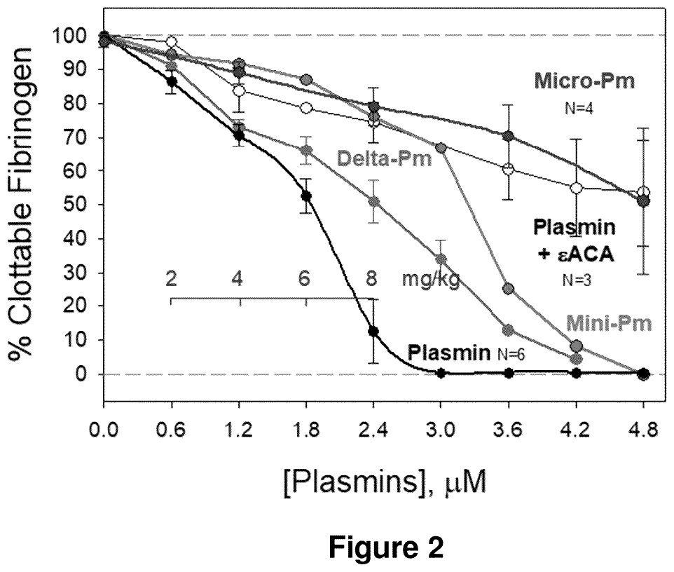

To determine the effect of different plasmin species on fibrinogen cleavage at different concentrations, an in-vitro study was executed. The various plasmin species tested are labelled beside the relevant plot in FIG. 2. The plasmin species was titrated into human plasma at the concentrations indicated, and residual fibrinogen content was assayed by the thrombin-coagulable fibrinogen assay reported by Blomback (Blombãck B., Blombãck M. Purification of human and bovine fibrinogen. Arkh. Kem. 10, 415, 1956). Briefly, the principle of this method is to isolate thrombin-coagulable material from citrated plasma. Once the clot is formed by the reaction of thrombin and fibrinogen, it is washed extensively and dissolved in alkaline urea for spectrophotometric measurement of fibrin concentration.

Experimental details: 20 μl of TBS was added to 230 μl citrated plasma; 10 μl of 0.239 mg/ml thrombin was added to clot plasma instantly (10 times diluted thrombin from Enzyme Research, 2.39 mg/ml, 1000 units). The tubes were vortexed after 3 min of incubation at 37° C. and centrifuged for 3 min at RT in a tabletop Eppendorf centrifuge at 14 000 rpm. Supernatant (serum) was removed and 1 ml of TBS is added to each tube. After intensive vortexing, the tubes were centrifuged again at 14 000 rpm. Supernatant was removed and 1 ml of 1M NaOH was added to each tube to dissolve the clots. The tubes were incubated at 70-80° C. for 2-4 minutes, cooled down and measured on the spectrophotometer for Absorbance at 280 nm.

Incubation with plasmin: (5 min at 37° C., varying amounts of plasmin as per FIG. 2) tubes with plasma are placed in the water bath at 37° C. Varying amounts of plasmin (0-20 μl of 5 mg/ml solution) are added to each tube with the time delay of 30 seconds. 5-min incubation is stopped by addition of 10 μl of thrombin. The rest of the assay is done as described above. Each concentration point is done in triplicate.

Full-length plasmin was prepared according to the procedures/methodology known by those skilled in the art and detailed in section 18.3 of Novokhatny, V. et al., Acid Stabilised Plasmin as a Novel Direct-Acting Thrombolytic, Ch 18, pg 259-271, Production of Plasma Proteins for Therapeutic Use, Eds. J. Bertolini, et al., Wiley, 2013 [Print ISBN:9780470924310|Online ISBN:9781118356807], the contents of which are incorporated herein by reference. Truncated plasmin mutants were prepared recombinantly using manufacturing processes within the common general knowledge of the skilled person.

The full-length plasmin utilised in all the experiments outlined herein was a mixture of predominantly Lys-plasmin with minor amounts of Glu-plasmin. Overtime, Lys-plasmin will convert any Glu-plasmin in the mixture to Lys-plasmin. By “full-length plasmin” the present specification means this mixture in unspecified proportions, but with Lys-plasmin in a vast excess.

From FIG. 2 it is evident that the addition of plasmin and its variants at increasingly higher concentrations resulted in decreasing concentrations of clottable fibrinogen in human plasma. Full-length/non-truncated plasmin at concentrations greater than 2.4 μM (8 mg/kg dose equivalent) completely depleted all clottable fibrinogen in the samples of human plasma.

The effect of plasmin on fibrinogen was seen most profoundly with the full-length plasmin. Truncated species of plasmin such as mini- or micro-plasmin exhibited a lesser effect on clottable fibrinogen levels in human plasma. The addition of ε-aminocaproic acid, a known inhibitor of kringle-mediated interactions in plasmin, resulted in the protease effect of plasmin on fibrinogen being greatly diminished (thin black line).

These in-vitro experiments with human plasma suggest that full-length plasmin could be used as a tool to control the level of circulating fibrinogen in human patients in a controllable fashion.

### Example 2: Ex-Vivo Viscosity Studies

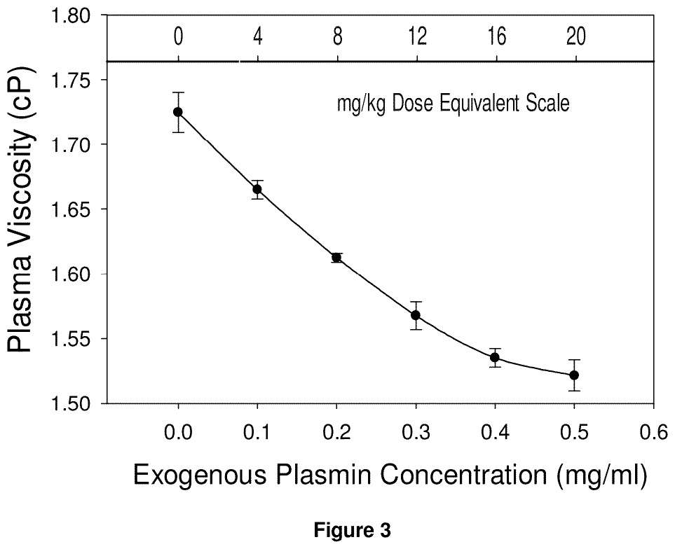

To test the effect of full-length plasmin on plasma viscosity, a series of experiments were conducted in which human pooled plasma was incubated for 5 minutes with increasing concentrations of plasmin. 360 μl of 2.78 mg/ml plasmin was added to 10 ml of the plasma and after 5 min incubation 1.5 ml aliquots were placed into Viscolab 4000 (Cambridge Viscosity) laboratory viscometer. The data expressed in centiPoise units were plotted versus plasmin concentration as shown in FIG. 3. As seen from FIG. 3, a significant decrease in viscosity from 1.725±0.015 to 1.63±0.0035 cP was observed.

The inserted scale on the top half of the graph provides an illustrative guide as to equivalency compared to animal dosing. For example, an 8 mg/kg dose used in a rat wound healing study would roughly be equivalent to ˜0.2 mg/ml plasma concentration of Plasmin assuming that blood volume of a 500 g rat is ˜35-40 ml, of which plasma is ˜20 ml.

### Example 3—Evaluation of the Effect of Systemic Administration of Plasmin in a Type 2 Diabetic Wound Healing Model

Male Zucker diabetic fatty rats (ZDF-Leprfa/Crl, obese), JVC-catheterized, were obtained from Charles River. The rats were 16 weeks of age and their weight was from 400 to 450 g. The rats had blood glucose values of >14 mmol/L or 252 mg/dl. The study was blinded for treatments during the in-life phase.

The rats were divided into three groups: Group 1-Normal Saline; Group 2-Recombinant Albumin; 2.8 mg/ml, 8 mg/kg; and Group 3-Full-Length Plasmin; 2.78 mg/ml, 8 mg/kg.

Each group consisted of 10 rats. Dose volume(s) of 3 mL/kg were utilised with a dose frequency of 10 days. The total study duration was 15 days.

The animals were anesthetized using a chamber with isoflurane/oxygen gas mixture. A metal rod (25 g, 1 cm in diameter) was heated to 95-100° C. by submersion in boiling water. The rod was immediately positioned vertically, for 6 seconds, without additional pressure on the back skin of a rat that had been depilated 3 days before wounding. After wounding, the rats were individually caged and the wounds were not dressed. Approximately 24 hours following the burn induction, 0.1 mL of the test items and reference items were administered intravenously once daily during the dosing period. During the study, assessments included mortality checks, clinical observations, and body weight evaluations.

Daily direct macroscopic wound observations were performed immediately following burn induction until the day of necropsy, with daily digital photos of the burn wounds with the surrounding cutaneous wound area. The outlines of the wound edge and areas covered with slough/eschar were drawn on sterile transparent sheets every 2 days (Days 1, 3, 5, 7, 9, 11, 13, and 15), and the areas contained inside the outlines were measured planimetrically by computer analysis. Blood samples were collected for clinical pathology (haematology and coagulation) from all animals at scheduled necropsy.

Macroscopic observations were performed on all pre-terminal and surviving animals at respective necropsies, and representative wound burn and/or unwounded skin samples were collected. Histopathological evaluations were performed on wound burn skin samples from 5 animals per group. In addition, wound burn skin and unwounded abdominal skin samples were collected from 5 animals per group and frozen in liquid nitrogen.

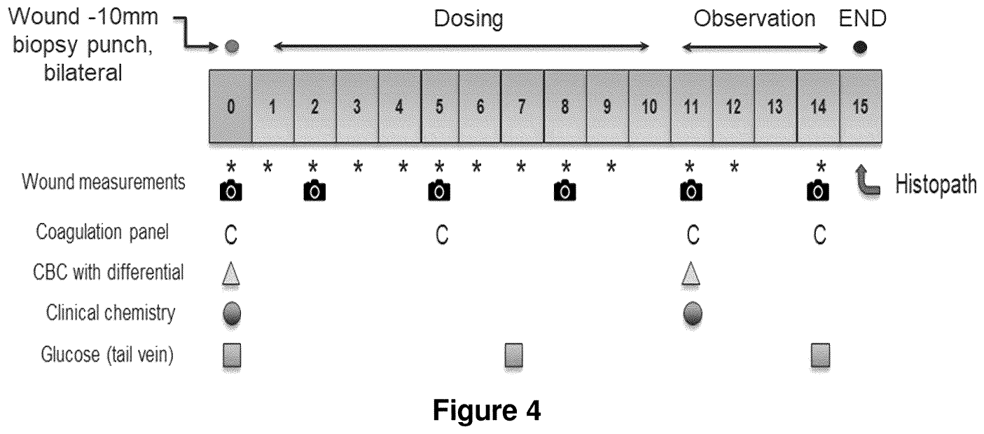

A schematic of the full test protocol is depicted in FIG. 4, and the analyses performed are outlined below.
1. Firstly, the Complete Blood Count (CBC) with differential was evaluated. Several parameters were studied: Red Blood Cell Count (RBC), Red Blood Cell Distribution Width (RDW), Mean Corpuscular Volume (MCV), Haematocrit, Haemoglobin (Hgb, Hb), Mean Corpuscular Haemoglobin (MCH), Mean Corpuscular Haemoglobin Concentration (MCHC), White Blood Cell Count (WBC), Percentage and Absolute Differential Counts/White Blood Cell Differential and Platelet Count.
2. Clinical chemistry parameters were studied such as: alanine aminotransferase (u/l), aspartate aminotransferase (u/l), alkaline phosphatase (u/l), blood urea nitrogen (mg/dl), total bilirubin (mg/dl), direct bilirubin (mg/dl), total protein (g/dl), albumin (g/dl), creatine (mg/dl), creatine kinase (u/l), cholesterol (mg/dl), triglycerides (mg/dl), glucose (mg/dl).
3. Finally, a coagulation panel of parameters were evaluated: prothrombin time (PT) (seconds), activated partial thromboplastin time (APTT) (seconds) and fibrinogen (FIB) (mg/dl).

### Example 4—Results from the Rat Wound Model in Example 3

Groups 1 to 3 showed very little difference between the parameters outlined in the previously mentioned points 1 and 2 paragraphs. Results not shown. However, with respect to blood fibrinogen levels Group 3 (Plasmin) showed a significant decrease in comparison to the control groups (Group 1 and 2).

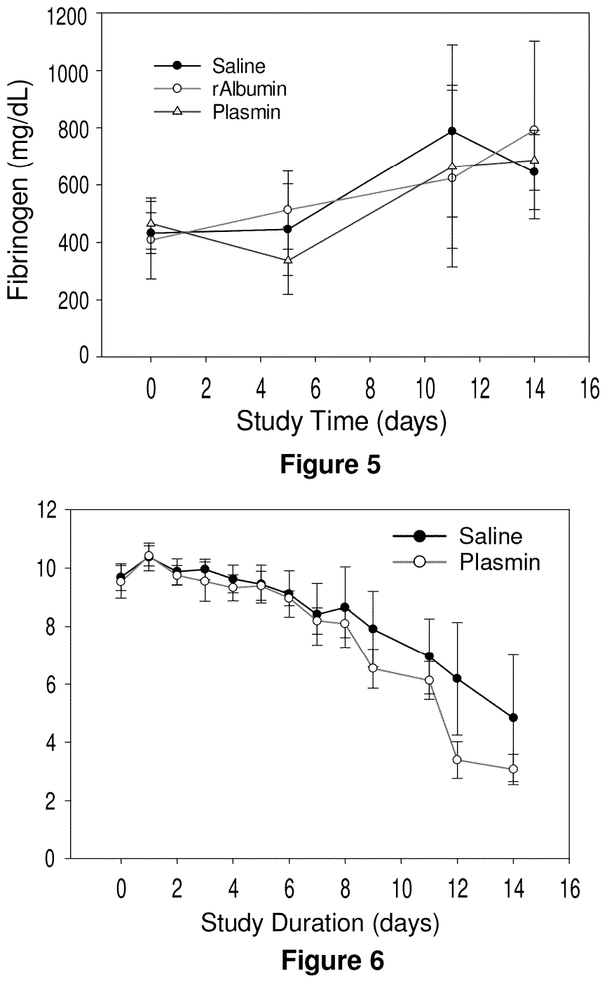

FIG. 5 plots the recorded fibrinogen levels (mg/dl) in the three groups over the study duration (n=5). Rats treated with full-length plasmin showed a reduction in fibrinogen concentrations throughout the study duration.

The mean (n=5) wound sizes for plasmin treated rats versus saline treated rats at the end of the study are tabulated in Table 2. Moreover, the results are plotted in FIG. 6.

TABLE 2

| | Group 1 - | Group 3 - | % | % |
|---|---|---|---|---|
| | Saline | Plasmin | smaller | Average |
| Day 12 | 6.19 | 3.39 | −45% | −40.5% |
| Day 14 | 4.83 | 3.07 | −36% | |

### Example 5—Sample Pathology

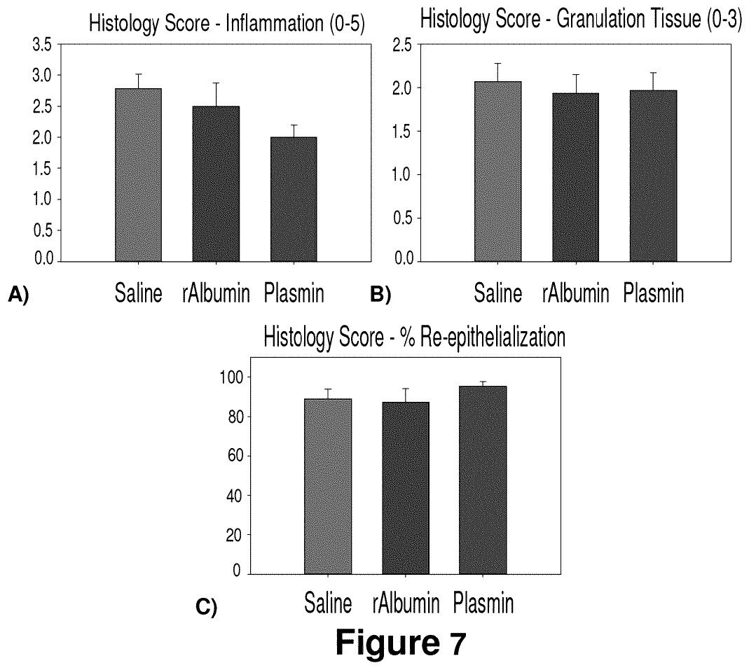

Two sections of wound sites from both sides of the site were assessed by Pathologist in a blind manner. The histology score criteria utilised is outlined below and the results are illustrated in FIG. 7.

Inflammation across the wound site is scored on a scale from 0-5 where
0. None,
1. Minimal—rare foci of inflammatory cells such as neutrophils, macrophages, and lymphocytes present across the wound bed; barely detectable,
2. Mild—a few foci of inflammatory cells across the wound bed noticeably above detectable levels
3. Moderate—obvious inflammatory cells in scattered to coalescing foci across the wound bed
4. Marked—near diffuse infiltrate of inflammatory cells affecting 50-75% of the wound bed
5. Severe—diffuse infiltrate of inflammatory cells affecting >75% of the wound bed.

Granulation tissue is scored on a scale from 0-3 where:
0. No granulation tissue present in the wound bed;
1. Minimal amounts of granulation tissue present in the wound bed;
2. Moderate amounts of granulation tissue present in the wound bed;
3. Robust granulation tissue is present in the wound bed.

Percentage of re-epithelialization is the approximate % of the defect to the nearest 5%, covered by an epithelial surface.

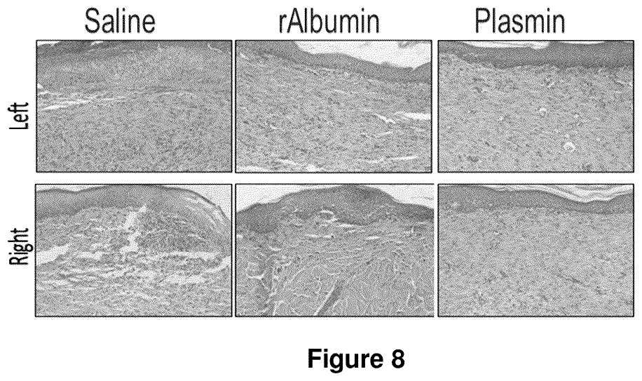

The results are plotted in FIGS. 7A-7C, and representative slides of wound sites are provided in FIG. 8. The results demonstrate that compared to control groups 1 and 2, inflammation in the full-length plasmin treated group was significantly reduced. Inflammation was predominantly composed of macrophages, lymphocytes, and plasma cells but neutrophils were present in areas of more severe inflammation in some animals that did not yet have complete re-epithelialization.

Granulation tissue and the percent re-epithelialization were not significantly different between the groups, but the plasmin treated groups did have a trend towards increased re-epithelialization compared to the control groups. This improvement was observed for both the left and right side with enhanced coverage of the wound bed by new epithelial tissue migrating across mature granulation tissue. In all groups, granulation tissue was prominent in the wound bed.

The above studies show that improved wound healing is seen in the full-length plasmin treat group, with an attendant ˜25% decrease in circulating plasma fibrinogen levels.

### Sequences

The sequences referred to in the preceding text are outlined below in fasta format. In the event of a discrepancy between the sequence listed in this text and the corresponding sequence in the accompanying sequence listing, the sequence listed in this text shall be the prevailing sequence for the purposes of correcting an error.

Human Plasminogen Protein Sequence
SEQ ID NO: 1
EPLDDYVNTQGASLFSVTKKQLGAGSIEECAAKCEEDEEFTCRAFQ
YHSKEQQCVIMAENRKSSIIIRMRDVVLFEKKVYLSECKTGNGKN
YRGTMSKTKNGITCQKWSSTSPHRPRFSPATHPSEGLEENYCRNP
DNDPQGPWCYTTDPEKRYDYCDILECEEECMHCSGENYDGKISKT
MSGLECQAWDSQSPHAHGYIPSKFPNKNLKKNYCRNPDRELRPWC
FTTDPNKRWELCDIPRCTTPPPSSGPTYQCLKGTGENYRGNVAVT
VSGHTCQHWSAQTPHTHNRTPENFPCKNLDENYCRNPDGKRAPWC
HTTNSQVRWEYCKIPSCDSSPVSTEQLAPTAPPELTPVVQDCYHG
DGQSYRGTSSTTTTGKKCQSWSSMTPHRHQKTPENYPNAGLTMNY
CRNPDADKGPWCFTTDPSVRWEYCNLKKCSGTEASVVAPPPVVLL
PDVETPSEEDCMFGNGKGYRGKRATTVTGTPCQDWAAQEPHRHSI
FTPETNPRAGLEKNYCRNPDGDVGGPWCYTTNPRKLYDYCDVPQC
AAPSFDCGKPQVEPKKCPGRVVGGCVAHPHSWPWQVSLRTRFGMH
FCGGTLISPEWVLTAAHCLEKSPRPSSYKVILGAHQEVNLEPHVQ
EIEVSRLFLEPTRKDIALLKLSSPAVITDKVIPACLPSPNYVVAD
RTECFITGWGETQGTFGAGLLKEAQLPVIENKVCNRYEFLNGRVQ
STELCAGHLAGGTDSCQGDSGGPLVCFEKDKYILQGVTSWGLGCA
RPNKPGVYVRVSRFVTWIEGVMRNN

1. A method for the treatment of a wound or ulcer in a patient in need thereof, the method comprising parenteral administration of a therapeutically effective amount of a fibrinogenase to the patient.

2. The method of claim 1, wherein the parenteral administration is an administration route selected from the group consisting of intravenous, intramuscular, intraperitoneal, and subcutaneous.

3. The method of claim 1, wherein the fibrinogenase is administered intravenously.

4. The method of claim 1, wherein the fibrinogenase is selected from the group consisting of an a-fibrinogenase, a b-fibrinogenase, a y-fibrinogenase, a metallo-a-fibrinogenase, allium-a-fibrinogenase, a plasmin, and combinations thereof.

5. The method of claim 1, wherein the fibrinogenase is a plasmin.

6. The method of claim 5, wherein the plasmin is selected from the group consisting of Glu-plasmin, Lys-plasmin, midi-plasmin, mini-plasmin, micro-plasmin, deltaplasmin, and combinations thereof.

7. The method of claim 6, wherein the plasmin is selected from the group consisting of Glu-plasmin, Lys-Plasmin, and a combination thereof.

8. The method of claim 1, wherein the fibrinogenase is administered over a dosing period, and at one or more time-points during the dosing period the patient's plasma viscosity decreases by at least about 1.0% compared to the patient's pre-treatment plasma viscosity level.

9. The method of claim 1, wherein the fibrinogenase is administered over a dosing period, and at one or more time-points during the dosing period the patient's plasma viscosity decreases in the range of about 1.0% to about 20.0% compared to the patient's pre-treatment plasma viscosity level.

10. The method of claim 1, wherein the patient is suffering from an ulcer and the ulcer is selected from the group consisting of a venous leg ulcer, a diabetic ulcer, a pressure ulcer, an ischemic ulcer, and combinations thereof.

11. The method of claim 1, wherein the fibrinogenase is administered to the patient in at least one dose of a concentration of from about 1 mg/kg to about 100 mg/kg.

12. The method of claim 1, wherein the fibrinogenase is administered at initial dose of about 3 mg/kg to about 30 mg/kg on day 1 of an administration period, followed by about 3 mg/kg to about 30 mg/kg per dose during a multiple dosing period.

13. The method of claim 12, wherein the multiple dosing period may comprise from about 3 to about 30 administrations up to a total cumulative dose.

14. The method of claim 12, wherein the multiple dosing period may be from about 1 to about 10 weeks in duration.

15. The method of claim 1, wherein the fibrinogenase is administered as a pharmaceutical composition, the pharmaceutical composition further comprising a pharmaceutically acceptable carrier, and optionally a pharmaceutically acceptable excipient.

16. The method of claim 15, wherein the fibrinogenase is a plasmin and the pharmaceutical composition has an acidic pH.

17-47. (canceled)

48. The method of claim 13, wherein the multiple dosing period may be from about 1 to about 10 weeks in duration.
